(19) United States
(12) Patent Application Publication
(10) Pub. No.: US 2008/0261317 A1
(43) Pub. Date: Oct. 23, 2008

(54) Methods of Detecting Myocardial Ischemia and Myocardial Infarction

(76) Inventors: Michael A. Fifer, Boston, MA (US); Marc S. Sabatine, Boston, MA (US); Robert Gerszten, Boston, MA (US)

Correspondence Address: EWARDS ANGELL PALMER & DODGE LLP, P.O. BOX 55874, BOSTON, MA, 02205, US

(21) Appl. No.: 11/661,451

(22) PCT Filed: Sep. 6, 2005
(86) PCT No.: PCT/US2005/031722

§ 371 Date: Mar. 6, 2008

Related U.S. Application Data
(60) Provisional application No. 60/607,675, filed on Sep. 7, 2004.

(51) Int. Cl. G01N 33/68; G01N 33/50

(52) U.S. Cl. 436/89; 436/129; 436/98; 436/94

(57) ABSTRACT

The disclosed methods address the identification of myocardial ischemia and myocardial infarction using metabolomics, as well as the identification of metabolic products whose differential expression over time is indicative of myocardial ischemia and/or myocardial infarction.

### CROSS-REFERENCE TO RELATED APPLICATIONS/PATENTS & INCORPORATION BY REFERENCE

This application claims priority to U.S. Provisional Application Ser. No. 60/607,675, filed on Sep. 7, 2004, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List before the paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by National Institutes of Health Grant Nos. F32 HL68455 and R01 HL072872 and National Institutes of Health/National Human Genome Research Institute Grant No. P20 CA96470-01.

### BACKGROUND OF THE INVENTION

Coronary artery disease is a leading cause of morbidity and mortality worldwide.¹ Recognition of myocardial ischemia is critical both for diagnosing coronary heart disease and for selecting and evaluating the response to therapeutic interventions. Currently, myocardial ischemia is diagnosed through a combination of a history consistent with typical angina pectoris and labile electrocardiographic ST-segment and T wave changes, occurring either spontaneously or upon provocation with exercise testing (Gibbons, R., et al. 2002 ACC/ACA guideline update for the management of patients with chronic stable angina: a report of the American College of Cardiology/American Heart Association Task Force on Practice Guidelines; available upon request to ACC; Braunwald, E., et al. 2002 ACC/ACA guideline update for the management of patients with unstable angina and non-ST-segment elevation myocardial infarction: a report of the American College of Cardiology/American Heart Association Task Force on Practice Guidelines; available upon request to ACC). This approach, however, is often unsatisfactory due to the transient nature of electrocardiographic changes, as well as the subjective nature of history taking, particularly in the growing diabetic and elderly populations in whom symptoms are often atypical. Exercise testing with myocardial perfusion imaging is relatively accurate, but adds over $2000 to the cost and is difficult to implement rapidly in settings such as the emergency department (Gibbons, R., et al. 1997 J Am Coll Cardiol 30:260-311; Ritchie, J. L., et al. 1995 J Nucl Cardiol. 2:172-92). Although several biomarkers accurately diagnose patients with irreversible injury secondary to myocardial infarction, none are suitable for detecting the more subtle insult of myocardial ischemia (Morrow, D. A., et al. 2003 Clin Chem 49:537-9).

Acute Myocardial Infarction (MI) is the leading cause of death in the United States, with 500,000 of the approximately 1.1 million attacks each year being fatal (Braunwald, E., et al. 2000 J Am Coll Cardiol 36:970-1062). The steep time-to-treatment benefit curve underlying current reperfusion strategies exemplifies how early, reliable diagnosis of acute coronary syndromes has acquired not only prognostic, but increasingly therapeutic importance (Fibrinolytic Therapy Trialists' (FTT) Collaborative Group 1994 Lancet 343:311-322; Morrison, L. J. et al. 2000 JAMA 283:2686-92; Bonnefoy, E., et al. 2002 Lancet 360:825-9; Cannon, C. P., et al. 2000 JAMA 283:2941-7; Neumann, F. J., et al. 2003 JAMA 290:1593-9). However, conventional evaluations based on symptoms, physical examination and electrocardiographic findings are often inconclusive, particularly in aging and diabetic populations with preexisting coronary artery disease. Furthermore, available serum markers of myocardial infarction such as the troponins have limited sensitivity and specificity in the first several hours following the onset of injury (Braunwald, E., et al. 2000 J Am Coll Cardiol 36:970-1062).

Recent advances in proteomic and metabolic profiling technologies have enhanced the feasibility of high throughput patient screening for the diagnosis of disease states (Nicholson, J. K., et al. 2003 Nat Rev Drug Discov 2:668-76). The profiling of low molecular-weight metabolic products is particularly relevant to exercise physiology and myocardial ischemia. Small biochemicals are the end result of the entire chain of regulatory changes that occur in response to physiological stressors, disease processes, or drug therapy. In addition to serving as biomarkers, circulating metabolic products may themselves participate as regulatory signals such as in the control of blood pressure (He, W., et al. 2004 Nature 429:188-93).

### SUMMARY OF THE INVENTION

Circulating metabolic products that change depending on the presence of myocardial ischemia and myocardial infarction have now been identified and characterized. Such products can therefore serve as targets for therapeutic intervention or as substrates for molecular imaging.

In one embodiment, the invention provides a method of detecting myocardial ischemia or myocardial infarction in a subject comprising detecting a change in the amount of at least one member of the group consisting of lactic acid, hypoxanthine, inosine, alanine, GABA, oxaloacetate, citrulline, argininosuccinate, uric acid, citric acid, uridine, phenylalanine, tryptophan, serine, hydroxyhippuric acid, aconitic acid, and a metabolic product thereof in a biological sample obtained from the subject, thereby detecting myocardial ischemia or early myocardial infarction in the subject. In another embodiment of the invention, the change comprises a decrease in the amount of at least one member of the group consisting of GABA, oxaloacetate, citrulline, argininosuccinate, uric acid, citric acid, tryptophan, serine, uridine, and a metabolic product thereof. In yet another embodiment of the invention, the change comprises an increase in the amount of at least one member of the group consisting of lactic acid, hypoxanthine, inosine, alanine, phenylalanine, hydroxyhippuric acid, aconitic acid, and a metabolic product thereof. In a further embodiment of the invention, the change comprises an increase in the amount of at least one member of the group consisting of hydroxyhippuric acid, aconitic acid, and a metabolic product thereof.

In another embodiment, the invention provides a method of detecting myocardial ischemia or myocardial infarction in a subject comprising detecting an increase in the amount of at least one member of the group consisting of lactic acid, hypoxanthine, inosine, alanine, phenylalanine, hydroxyhippuric acid, aconitic acid, and a metabolic product thereof and a decrease in at least one member of the group consisting of GABA, oxaloacetate, citrulline, argininosuccinate, uric acid, citric acid, tryptophan, serine, uridine, and a metabolic product thereof.

In one embodiment of the invention, myocardial infarction is detected. The myocardial infarction can be early myocardial infarction. In another embodiment of the invention, early myocardial infarction corresponds to that early period of mycardial infarction during which standard markers such as troponins are not effective for detection or diagnosis. In yet another embodiment of the invention, early myocardial infarction corresponds to within two hours of onset of myocardial infarction. In another embodiment, the invention provides a method of detecting myocardial infarction in a subject comprising detecting a change comprising an increase in the amount of at least one member of the group consisting of lactic acid, hypoxanthine, inosine, alanine, phenylalanine, hydroxyhippuric acid, aconitic acid, and a metabolic product thereof and a decrease in at least one member of the group consisting of GABA, citric acid, oxaloacetate, citrulline, argininosuccinate, uric acid, tryptophan, serine, uridine, and a metabolic product thereof. In another embodiment of the invention, the change comprises an increase in the amount of at least one member of the group consisting of hydroxyhippuric acid, aconitic acid, and a metabolic product thereof and a decrease in at least one member of the group consisting of GABA, citric acid, oxaloacetate, citrulline, argininosuccinate, uric acid, tryptophan, serine, uridine, and a metabolic product thereof.

In one embodiment of the invention, myocardial ischemia is detected. In another embodiment, the invention provides a method of detecting myocardial ischemia in a subject comprising detecting a change comprising an increase in the amount of at least one member of the group consisting of lactic acid, hypoxanthine, inosine, alanine, hydroxyhippuric acid, and a metabolic product thereof and a decrease in the amount of at least one member of the group consisting of GABA, oxaloacetate, citrulline, argininosuccinate, uric acid, citric acid, and a metabolic product thereof. In another embodiment of the invention, the change comprises an increase in the amount of at least one member of the group consisting of hydroxyhippuric acid and a metabolic product thereof and a decrease in at least one member of the group consisting of GABA, citric acid, oxaloacetate, citrulline, argininosuccinate, uric acid, and a metabolic product thereof.

In one embodiment of the invention, the biological sample comprises a blood sample or a preparation thereof. The preparation may comprise plasma or serum. In another embodiment of the invention, the subject is a human.

In one embodiment of the invention, the change is detected after administration of a controlled ischemic insult or planned myocardial infarction to the subject. The controlled ischemic insult may comprise exercise testing, and the planned myocardial infarction may comprise alcohol septal ablation for hypertrophic cardiomyopathy.

In one embodiment of the invention, the detecting comprises analyzing the sample, or a preparation thereof, using liquid chromatography and mass spectrometry. The mass spectrometry may comprise high sensitivity electrospray mass spectrometry.

In one embodiment, the invention provides a metabolic profile indicating myocardial ischemia or myocardial infarction in a subject comprising a change in the amount of at least one member of the group consisting of lactic acid, hypoxanthine, inosine, alanine, GABA, oxaloacetate, citrulline, argininosuccinate, uric acid, citric acid, uridine, phenylalanine, tryptophan, serine, hydroxyhippuric acid, aconitic acid, and a metabolic product thereof in a biological sample obtained from the subject.

In another embodiment, the invention provides a profile indicating myocardial ischemia or myocardial infarction in a subject comprising an increase in the amount of at least one member of the group consisting of lactic acid, hypoxanthine, inosine, alanine, phenylalanine, hydroxyhippuric acid, aconitic acid, and a metabolic product thereof and a decrease in the amount of at least one member of the group consisting of GABA, oxaloacetate, citrulline, argininosuccinate, uric acid, citric acid, tryptophan, serine, uridine, and a metabolic product thereof.

In another embodiment, the invention provides a profile indicating myocardial infarction in a subject comprising a change comprising an increase in the amount of at least one member of the group consisting of lactic acid, hypoxanthine, inosine, alanine, phenylalanine, hydroxyhippuric acid, aconitic acid, and a metabolic product thereof and/or a decrease in the amount of at least one member of the group consisting of GABA, citric acid, oxaloacetate, citrulline, argininosuccinate, uric acid, tryptophan, serine, uridine, and a metabolic product thereof. In another embodiment, the change comprises an increase in the amount of at least one member of the group consisting of hydroxyhippuric acid, aconitic acid, and a metabolic product thereof and a decrease in at least one member of the group consisting of GABA, citric acid, oxaloacetate, citrulline, argininosuccinate, uric acid, tryptophan, serine, uridine, and a metabolic product thereof.

In another embodiment, the invention provides a profile indicating myocardial ischemia in a subject comprising a change comprising an increase in the amount of at least one member of the group consisting of lactic acid, hypoxanthine, inosine, alanine, hydroxyhippuric acid, and a metabolic product thereof and/or a decrease in the amount of at least one member of the group consisting of GABA, oxaloacetate, citrulline, argininosuccinate, uric acid, citric acid, and a metabolic product thereof. In another embodiment of the invention, the change comprises an increase in the amount of at least one member of the group consisting of hydroxyhippuric acid and a metabolic product thereof and a decrease in at least one member of the group consisting of GABA, citric acid, oxaloacetate, citrulline, argininosuccinate, uric acid, and a metabolic product thereof.

In one embodiment of the profile of the invention, the change results from administration of a controlled ischemic insult or planned myocardial infarction to the subject. The controlled ischemic insult may comprise exercise testing, and the planned myocardial infarction may comprise alcohol septal ablation for hypertrophic cardiomyopathy.

In one embodiment, the invention provides a method of obtaining a metabolic profile of a subject afflicted with, or at risk of becoming afflicted with, myocardial ischemia, comprising the steps of:
i) analyzing a biological sample obtained from the subject; and
ii) detecting a change in the amount of at least one member of the group consisting of lactic acid, hypoxanthine, inosine, alanine, GABA, oxaloacetate, citrulline, argininosuccinate, uric acid, citric acid, hydroxyhippuric acid, and a metabolic product thereof,
   thereby obtaining a metabolic profile of a subject afflicted with, or at risk of becoming afflicted with, myocardial ischemia. In another embodiment of the invention, the change is in the amount of at least one member of the group consisting of hydroxyhippuric acid and a metabolic product thereof. In another embodiment of the invention, the biological sample is obtained from the subject before and after subjecting the subject to controlled ischemic insult. The controlled ischemic insult comprises exercise testing.

In another embodiment, the invention provides a method of obtaining a metabolic profile of a subject afflicted with, or at risk of becoming afflicted with, myocardial infarction, comprising the steps of:
i) analyzing a biological sample obtained from the subject; and
ii) detecting a change in the amount of lactic acid, hypoxanthine, inosine, alanine, GABA, oxaloacetate, citrulline, argininosuccinate, uric acid, citric acid, uridine, phenylalanine, tryptophan, serine, hydroxyhippuric acid, aconitic acid, and a metabolic product thereof,
   thereby obtaining a metabolic profile of a subject afflicted with, or at risk of becoming afflicted with myocardial infarction. In another embodiment of the invention, the change is in the amount of at least one member of the group consisting of hydroxyhippuric acid, aconitic acid, and a metabolic product thereof. In another embodiment of the invention, the biological sample is obtained before and after subjecting the subject to planned myocardial infarction. In one embodiment of the invention, the planned myocardial infarction comprises alcohol septal ablation for hypertrophic cardiomyopathy.

In one embodiment, the invention provides a method of identifying a metabolic biomarker for myocardial ischemia, comprising the steps of:
i) obtaining a biological sample from a subject before and after subjecting the subject to controlled ischemic insult;
ii) analyzing the samples for changes in amounts of metabolic products; and
iii) identifying the metabolic products, thereby identifying a metabolic biomarker for myocardial ischemia. The controlled ischemic insult may comprise exercise testing.

In another embodiment, the invention provides a method of identifying a metabolic biomarker for myocardial infarction, comprising the steps of:
i) obtaining a biological sample from a subject before and after subjecting the subject to planned myocardial infarction;
ii) analyzing the samples for changes in amounts of metabolic products; and
iii) identifying the metabolic products, thereby identifying a metabolic biomarker for myocardial infarction. The planned myocardial infarction may comprise alcohol septal ablation for hypertrophic cardiomyopathy. In one embodiment of the method of the invention, the analyzing comprises subjecting the sample, or a preparation thereof, to liquid chromatography and mass spectrometry, and wherein the identifying comprises comparing the mass spectra obtained with those of known metabolic products.

Other aspects of the invention are described in or are obvious from the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, in which:

FIG. 1 shows an X-Y scatterplot of the statistical significance of the change in metabolite levels from baseline to immediately post-exercise testing. The position on the X-axis represents the statistical significance of the change in controls, and the position on the Y-axis represents the statistical significance of the change in cases. Metabolites whose concentration changed significantly (P<0.05) after stress testing in either cases or controls are shown as colored circles, the rest as black dots. Red indicates the concentration of the metabolite increased, green that it decreased. The color of the rim of the circle indicates the direction of the change in controls, while the center indicates the direction of the change in cases. Some of the low molecular weight peaks seen reproducibly in human plasma have not yet been unambiguously identified, and are designated as such by the prefix MET.

FIG. 2 shows graphs depicting median and interquartile ranges of normalized log metabolite levels in patients with ischemia (closed squares) and in those without (open squares) at all three timepoints (baseline, immediately after stress testing, and four hours after stress testing) for lactic acid, GABA, and MET193. Degree of statistical significance for the change compared to baseline levels is indicated by * (P<0.05), ** (0.01), or *** (P<0.001). Degree of statistical significance for the comparison between the change in cases vs. controls is indicated by \ (P0.05), \\ (P>0.01), or \\\ (P>0.001).

FIG. 3 shows box and whisker plots of the changes seen immediately after stress testing in 6 metabolites in cases and controls. The line in the box represents the median change in the normalized log value, the boundaries of the box represent the 25^{th} and 75^{th} percentiles, the whiskers represent the 5^{th} and 95^{th} percentiles, and open circles represent the outliers. For each metabolte, P values shown below cases and controls indicate significance of the change from baseline. P values shown at top measure the significance in the difference in change between cases and controls.

FIG. 4 depicts, in bar graph form, the proportion of patients with inducible ischemia among patients categorized by the metabolic risk score.

FIG. 5 lists, in tabular form, the known metabolites analyzed herein.

FIG. 6 shows box and whisker plots of time (X axis) vs. intensity (of change in the amount of metabolite) (Y axis) for the seven most changed metabolites in the discovery cohort.

FIG. 7 shows a box and whisker plot of time (X axis) vs. intensity (of change in the amount of metabolite) (Y axis-“V₂₇₀”) to depict the performance of the Comp7 biomarker in the discovery cohort.

FIG. 8 shows a box and whisker plot of time (X axis) vs. intensity (of change in the amount of metabolite) (Y axis-“V₃₀₂”) to depict the performance of the Comp7 biomarker in the validation cohort.

FIG. 9 shows a box and whisker plot of time (X axis) vs. intensity (of change in the amount of metabolite) (Y axis-“V₄₇₇”) to depict the performance of the Comp7 biomarker in controls (stable coronary artery disease) vs. cases (acute myocardial infarction).

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Lackie and Dow, The Dictionary of Cell & Molecular Biology (3^{rd} ed. 1999); Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “myocardial ischemia” refers to a disorder of cardiac function caused by insufficient blood flow to the muscle tissue of the heart. The decreased blood flow may, for example, be due to narrowing of the coronary arteries (coronary arteriosclerosis), to obstruction by a thrombus (coronary thrombosis), or less commonly, to diffuse narrowing of arterioles and other small vessels within the heart. Severe interruption of the blood supply to the myocardial tissue may result in necrosis of cardiac muscle (myocardial infarction).

As used herein, “myocardial infarction (MI)” refers to the irreversible necrosis of heart muscle secondary to prolonged ischemia. This usually results from an imbalance of oxygen supply and demand. The appearance of cardiac enzymes in the circulation generally indicates myocardial necrosis.

As used herein, “metabolite” refers to any substance produced or used during all the physical and chemical processes within the body that create and use energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g. metabolite, metabolic precursor).

As used herein, “biological sample” refers to a sample obtained from a subject. The biological sample can be selected, without limitation, from the group consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, noncellular portion of the blood, distinguished from the serum obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female.

As used herein, “treatment” refers to ameliorating an adverse heart condition such as myocardial ischemia or myocardial infarction.

As used herein, “detecting” refers to methods which include identifying the presence or absence of substance(s) in the sample, quantifying the amount of substance(s) in the sample, and/or qualifying the type of substance. “Detecting” likewise refers to methods which include identifying the presence or absence of myocardial ischemia or early myocardial infraction in a subject.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

The terms “comprises”, “comprising”, and the like are intended to have the broad meaning ascribed to them in U.S. Patent Law and can mean “includes”, “including” and the like.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a”, “an”, and “the” include plural referents unless the context clearly indicates otherwise.

### II. Embodiments of The Invention

### Sample Collection and Preparation

In one embodiment of the invention, the subject may undergo exercise testing after initial sample collection and before subsequent sample collection. In another embodiment of the invention, the subject may undergo a planned heart attack after initial sample collection and before subsequent sample collection.

In one embodiment of the invention, samples may be collected from individuals over a longitudinal period of time. Obtaining numerous samples from an individual over a period of time can be used to verify results from earlier detections and/or to identify an alteration in polypeptide pattern as a result of, for example, pathology.

In one embodiment of the invention, the samples are analyzed without additional preparation and/or separation procedures.

In another embodiment of the invention, sample preparation and/or separation can involve, without limitation, any of the following procedures, depending on the type of sample collected and/or types of metabolic products searched: removal of high abundance polypeptides (e.g., albumin, and transferrin); addition of preservatives and calibrants, desalting of samples; concentration of sample substances; protein digestions; and fraction collection. In yet another embodiment of the invention, sample preparation techniques concentrate information-rich metabolic products and deplete polypeptides or other substances that would carry little or no information such as those that are highly abundant or native to serum.

In another embodiment of the invention, sample preparation takes place in a manifold or preparation/separation device. Such a preparation/separation device may, for example, be a microfluidics device. In yet another embodiment of the invention, the preparation/separation device interfaces directly or indirectly with a detection device. Such a preparation/separation device may, for example, be a fluidics device.

In another embodiment of the invention, the removal of undesired polypeptides (e.g., high abundance, uninformative, or undetectable polypeptides) can be achieved using high affinity reagents, high molecular weight filters, column purification, ultracentrifugation and/or electrodialysis. High affinity reagents include antibodies that selectively bind to high abundance polypeptides or reagents that have a specific pH, ionic value, or detergent strength. High molecular weight filters include membranes that separate molecules on the basis of size and molecular weight. Such filters may further employ reverse osmosis, nanofiltration, ultrafiltration and microfiltration.

Ultracentrifugation constitutes another method for removing undesired polypeptides. Ultracentrifugation is the centrifugation of a sample at about 60,000 rpm while monitoring with an optical system the sedimentation (or lack thereof) of particles. Finally, electrodialysis is an electromembrane process in which ions are transported through ion permeable membranes from one solution to another under the influence of a potential gradient. Since the membranes used in electrodialysis have the ability to selectively transportions having positive or negative charge and reject ions of the opposite charge, electrodialysis is useful for concentration, removal, or separation of electrolytes.

In another embodiment of the invention, the manifold or microfluidics device performs electrodialysis to remove high molecular weight polypeptides or undesired polypeptides. Electrodialysis can be used first to allow only molecules under approximately 30 kD to pass through into a second chamber. A second membrane with a very small molecular weight (roughly 500 D) allows smaller molecules to egress the second chamber.

Upon preparation of the samples, metabolic products of interest may be separated in another embodiment of the invention. Separation can take place in the same location as the preparation or in another location. In one embodiment of the invention, separation occurs in the same microfluidics device where preparation occurs, but in a different location on the device. Samples can be removed from an initial manifold location to a microfluidics device using various means, including an electric field. In another embodiment of the invention, the samples are concentrated during their migration to the microfluidics device using reverse phase beads and an organic solvent elution such as 50% methanol. This elutes the molecules into a channel or a well on a separation device of a microfluidics device.

Chromatography constitutes another method for separating subsets of substances. Chromatography is based on the differential absorption and elution of different substances. Liquid chromatography (LC), for example, involves the use of fluid carrier over a non-mobile phase. Conventional LC columns have an in inner diameter of roughly 4.6 mm and a flow rate of roughly 1 ml/min. Micro-LC has an inner diameter of roughly 1.0 mm and a flow rate of roughly 40 ul/min. Capillary LC utilizes a capillary with an inner diameter of roughly 300 im and a flow rate of approximately 5 ul/min. Nano-LC is available with an inner diameter of 50 um-1 mm and flow rates of 200 nl/min. The sensitivity of nano-LC as compared to HPLC is approximately 3700 fold. Other types of chromatography contemplated for additional embodiments of the invention include, without limitation, thin-layer chromatography (TLC), reverse-phase chromatography, high-performance liquid chromatography (HPLC), and gas chromatography (GC).

In another embodiment of the invention, the samples are separated using capillary electrophoresis separation. This will separate the molecules based on their electrophoretic mobility at a given pH (or hydrophobicity).

In another embodiment of the invention, sample preparation and separation are combined using microfluidics technology. A microfluidic device is a device that can transport liquids including various reagents such as analytes and elutions between different locations using microchannel structures.

### Detection

In one embodiment of the invention, the sample may be delivered directly to the detection device without preparation and/or separation beforehand. In another embodiment of the invention, once prepared and/or separated, the metabolic products are delivered to a detection device, which detects them in a sample. In another embodiment of the invention, metabolic products in elutions or solutions are delivered to a detection device by electrospray ionization (ESI). In yet another embodiment of the invention, nanospray ionization (NSI) is used. Nanospray ionization is a miniaturized version of ESI and provides low detection limits using extremely limited volumes of sample fluid.

In another embodiment of the invention, separated metabolic products are directed down a channel that leads to an electrospray ionization emitter, which is built into a microfluidic device (an integrated ESI microfluidic device). Such integrated ESI microfluidic device may provide the detection device with samples at flow rates and complexity levels that are optimal for detection. Furthermore, a microfluidic device may be aligned with a detection device for optimal sample capture.

Detection devices can comprise of any device or experimental methodology that is able to detect metabolic product presence and/or level, including, without limitation, IR (infrared spectroscopy), NMR (nuclear magnetic resonance), including variations such as correlation spectroscopy (COSY), nuclear Overhauser effect spectroscopy (NOESY), and rotating frame nuclear Overhauser effect spectroscopy (ROESY), and Fourier Transform, 2-D PAGE technology, Western blot technology, tryptic mapping, in vitro biological assay, immunological analysis, LC-MS (liquid chromatography-mass spectrometry), and MS (mass spectrometry).

For analysis relying on the application of NMR spectroscopy, the spectroscopy may be practiced as one-, two-, or multidimensional NMR spectroscopy or by other NMR spectroscopic examining techniques, among others also coupled with chromatographic methods (for example, as LC-NMR). In addition to the determination of the metabolic product in question, ¹H-NMR spectroscopy offers the possibility of determining further metabolic products in the same investigative run. Combining the evaluation of a plurality of metabolic products in one investigative run can be employed for so-called “pattern recognition”. In one embodiment of the invention, the strength of diagnostic statements which the methods permit is improved by an evaluation in the pattern recognition mode as compared to the isolated determination of the concentration of one metabolic product.

For immunological analysis, for example, the use of immunological reagents (e.g. antibodies), generally in conjunction with other chemical and/or immunological reagents, induces reactions or provides reaction products which then permit detection and measurement of the whole group, a subgroup or a subspecies of the metabolic product(s) of interest. These immunological methods according to the invention may be carried out in practice along the lines of the method published by Smal and Baldo (Smal, M. A. et al. 1991, Lipid 26: 1130-1135; Baldo, B. A. et al. 1991, Lipids 26: 1136-1139). Reference is made to these publications.

In one embodiment of the invention, mass spectrometry is relied upon to detect metabolic products present in a given sample. In another embodiment of the invention, an ESI-MS detection device. Such an ESI-MS may utilizes a time-of-flight (TOF) mass spectrometry system. Quadrupole mass spectrometry, ion trap mass spectrometry, and Fourier transform ion cyclotron resonance (FTICR-MS) are likewise contemplated in additional embodiments of the invention.

In another embodiment of the invention, the detection device interfaces with a separation/preparation device or microfluidic device, which allows for quick assaying of many, if not all, of the metabolic products in a sample. A mass spectrometer may be utilized that will accept a continuous sample stream for analysis and provide high sensitivity throughout the detection process (e.g., an ESI-MS). In another embodiment of the invention, a mass spectrometer interfaces with one or more electrosprays, two or more electrosprays, three or more electrosprays or four or more electrosprays. Such electrosprays can originate from a single or multiple microfluidic devices.

In another embodiment of the invention, the detection system utilized allows for the capture and measurement of most or all of the metabolic products introduced into the detection device.

In another embodiment of the invention, the detection system allows for the detection of change in a defined combination (“composite”) of metabolic products.

### Signal Processing

In another embodiment of the invention, the output from a detection device can subsequently be processed, stored, and further analyzed or assayed using a bio-informatics system. A bio-informatics system may include one or more of the following, without limitation: a computer; a plurality of computers connected to a network; a signal processing tool(s); a pattern recognition tool(s); a tool(s) to control flow rate for sample preparation, separation, and detection.

The data processing utilizes mathematical foundations. In another embodiment of the invention, dynamic programming is used to align a separation axis with a standard separation profile. Intensities may be normalized, for example, by fitting roughly 90% of the intensity values into a standard spectrum. The data sets can then be fitted using wavelets designed for separation and mass spectrometer data. In yet another embodiment of the invention, data processing filters out some of the noise and reduces spectrum dimensionality, potentially allowing for pattern recognition.

Following data processing, pattern recognition tools can be utilized to identify subtle differences between phenotypic states. Pattern recognition tools are based on a combination of statistical and computer scientific approaches, which provide dimensionality reduction. Such tools are scalable.

### Kits

In another embodiment, the invention provides kits for monitoring and diagnosing myocardial ischemia or early myocardial infarction, wherein the kits can be used to detect the metabolic products described herein. For example, the kits can be used to detect any one or more of the metabolic products potentially differentially present in samples of the subjects before vs. after the administration of a controlled insult.

The kits of the invention may include instructions for the assay, reagents, testing equipment (test tubes, reaction vessels, needles, syringes, etc.), standards for calibrating the assay, and/or equipment provided or used to conduct the assay. The instructions provided in a kit according to the invention may be directed to suitable operational parameters in the form of a label or a separate insert.

This invention is further illustrated by the following examples, which should not be construed as limiting. A skilled artisan should readily understand that other similar instruments with equivalent function/specification, either commercially available or user modified, are suitable for practicing the instant invention. Rather, the invention should be construed to include any and all applications provided herein and all equivalent variations within the skill of the ordinary artisan.

### II. Examples

Metabolic profiling technologies were applied using liquid chromatography coupled with high sensitivity electrospray mass spectrometry, to blood samples obtained from patients undergoing exercise stress testing. This approach is particularly powerful as serial sampling can be performed in patients before and after a controlled ischemic insult, thereby allowing each patient to serve as his or her own biological control.

### Example 1

### Ischemic Insult

### Change in Metabolite Levels Before Vs. after Exercise

A. Patients

Patients who underwent stress testing with myocardial perfusion imaging at Brigham and Women's Hospital and Massachusetts General Hospital were enrolled in a prospective biomarker cohort study. The Human Research Committee approved the study protocol and all patients provided written informed consent. All patients who were referred for stress testing for the evaluation of possible myocardial ischemia were eligible for participation. Patients who underwent pharmacologic testing were excluded. For these analyses, blood samples from a total of 36 patients, 18 with evidence of inducible ischemia (hereinafter referred to as “cases”) and 18 without evidence of ischemia (hereinafter referred to as “controls”), were selected for metabolic profiling.

B. Study Protocol

Data were obtained on each patient's age, sex, race, weight cardiac risk factors (including hypertension, diabetes mellitus, smoking, and hyperlipidemia), prior cardiac disease [including angina, myocardial infarction (MI), congestive heart failure (CHF), angiographically confirmed significant coronary artery disease (CAD), percutaneous coronary intervention, and coronary artery bypass grafting (CABG)], and cardiac medications.

Thus, the baseline characteristics and stress test performance parameters for these patients are listed in Table 1, below.

TABLE 1
Patient characteristics (with data are presented
as mean ± SD or number (%) of patients).

| | |
|---|---|
| No ischemia | Ischemia |
| (n = 18) | (n = 18) |

Demographics

| Age (years) | 64 ± 10 | | 65 ± 11 | |
|---|---|---|---|---|
| Male | 9 | (50%) | 15 | (83%) |
| White | 12 | (67%) | 16 | (89%) |

Cardiac risk factors

| | | | | |
|---|---|---|---|---|
| Hypertension | 14 | (78%) | 13 | (72%) |

Diabetes

| Insulin-dependent | 0 | | 4 | (22%) |
|---|---|---|---|---|
| Non-insulin dependent | 5 | (28%) | 5 | (28%) |
| None | 13 | (72%) | 9 | (50%) |

Smoking

| Current | 0 | | 1 | (6%) |
|---|---|---|---|---|
| Former | 7 | (39%) | 13 | (72%) |
| Never | 11 | (61%) | 4 | (22%) |
| Hyperlipidemia | 11 | (61%) | 14 | (78%) |

| | | |
|---|---|---|
| # of cardiac risk factors. | 2.1 ± 0.9 | 3.0 ± 0.9 |

Prior cardiovascular disease

| | | | | |
|---|---|---|---|---|
| Coronary artery disease | 5 | (28%) | 15 | (83%) |
| Myocardial infarction | 4 | (22%) | 13 | (72%) |
| Coronary revascularization | 5 | (28%) | 12 | (67%) |
| Congestive heart failure | 0 | (0%) | 5 | (28%) |
| Peripheral arterial disease | 0 | (0%) | 3 | (17%) |

Stress test parameters

| Duration (mins) | 8.8 ± 2.3 | | 6.8 ± 2.2 | |
|---|---|---|---|---|
| Metabolic equivalents (METs) | 10.0 ± 2.6 | | 7.9 ± 2.7 | |
| Chest pain | 7 | (39%) | 9 | (50%) |
| ST deviation >_1 mm | 2 | (12%) | 10 | (56%) |

| | | |
|---|---|---|
| Percentage of myocardium | 3 ± 4 | 28 ± 12 defect |
| with any perfusion | | (mean ± SD) |
| Percentage of myocardium | 0 ± 0 | 17 ± 8 perfusion defect |
| with reversible | | (mean ± SD) |

The mean ages of the two groups were comparable, though, as expected, patients with inducible ischemia had slightly more cardiac risk factors (3.0±0.9 vs. 2.1=0.9) and were more likely to have a documented history of coronary disease.

Patients underwent exercise testing using the standard Bruce protocol (Fletcher, G. F., et al. 2001 Circulation 104:1694-1740). Symptoms, heart rate, blood pressure, and a 12-lead ECG were recorded before the test, midway through each stage, and during recovery.

The stress test was terminated if there was physical exhaustion, severe angina, >2 mm horizontal or downsloping ST-segment depression, >20 mm Hg fall in systolic blood pressure, or sustained ventricular arrhythmia. Duration of the stress test, metabolic equivalents (METs) achieved, peak heart rate, and peak blood pressure were recorded. If the patient developed angina during the test, the timing, quality (typical vs. atypical), and effect on the test (limiting or non-limiting) were noted. The maximal horizontal or downsloping ST segment changes were recorded in each ECG lead.

C. SPECT Myocardial Perfusion Imaging

A stress-rest imaging protocol was used. 99Tc tetrofosmin was administered at peak stress and imaging was performed soon thereafter. Four hours later, a second injection was administered and repeat imaging was performed. Quantitative analysis of perfusion was performed using the CEqual method to calculate the percent reversible and fixed perfusion defects (Garcia, E. V., et al. 1990 Am J Cardiol 66:23 E-31E). Patients with >5% reversible perfusion defect were selected as cases and those without any perfusion defect were selected as controls. Left ventricular ejection fraction was calculated using commercially available software (DePuey, E. G., et al. 1993 J Nucl Med 34:1871-6).

In the 18 cases, all subjects had reversible perfusion defects and the mean percentage of myocardium with a reversible perfusion defect was 17±8%, whereas, by definition, no controls had any degree of a reversible perfusion defect. Although coronary angiography was not mandated by the protocol of this study, 14 of the 18 cases did undergo coronary angiography and all 14 had angiographic confirmation of multivessel or severe complex single-vessel coronary artery disease.

D. Metabolic Profiling—HPLC and Mass Spectrometry Analysis

Blood samples were obtained immediately before, immediately after, and 4 hours after stress testing. Blood samples were placed on ice and processed within 60 minutes. Citrate-anticoagulated plasma was stored at −80° C., and aliquots were thawed for these analyses. Amino acids and amines were separated on a Luna phenyl-hexyl column (Phenomenex, Torrance, Calif.) under reverse phase chromatography using acetonitrile/water/0.1% acetic acid at pH 3.5-4.0 in a run time of 1.5 minutes. Sugars and ribonucleotides were separated on a Luna amino column (Phenomenex, Torrance, Calif.) under normal phase using acetonitrile/water/0.25% ammonium hydroxide/10 mM ammonium acetate at pH 11 in a run time of 3.5 minutes.

Organic acids were separated using a Synergi Polar-RP column (Phenomenex, Torrance, Calif.) under reverse phase using acetonitrile/water/5 mM ammonium acetate at pH 5.6-6.0 in a run time of 3.5 minutes. Columns were connected in parallel via an automated switching valve on a robotic sample loader (Leap Technologies). A triple quadrupole mass spectrometer (API4000, Applied Biosystem/Sciex) was operated in an automated switching polarity mode using a turbo ion spray LC/MS interface under selected reaction monitoring (SRM) conditions. A total of 477 parent/daughter (P/D) ion pairs were monitored through six SRM experiments on each sample.

Peak areas for each parent/daughter pair were integrated and analytes with areas below the limit of detection of the LC/MS/MS were dropped from further analysis. Peak area ratios to an internal standard were computed to normalize variation in injection volume. The peak area ratios were then log transformed and log peak area ratios per sample were normalized by subtracting the median of all analytes to account for sample-sample variation in blood concentration.

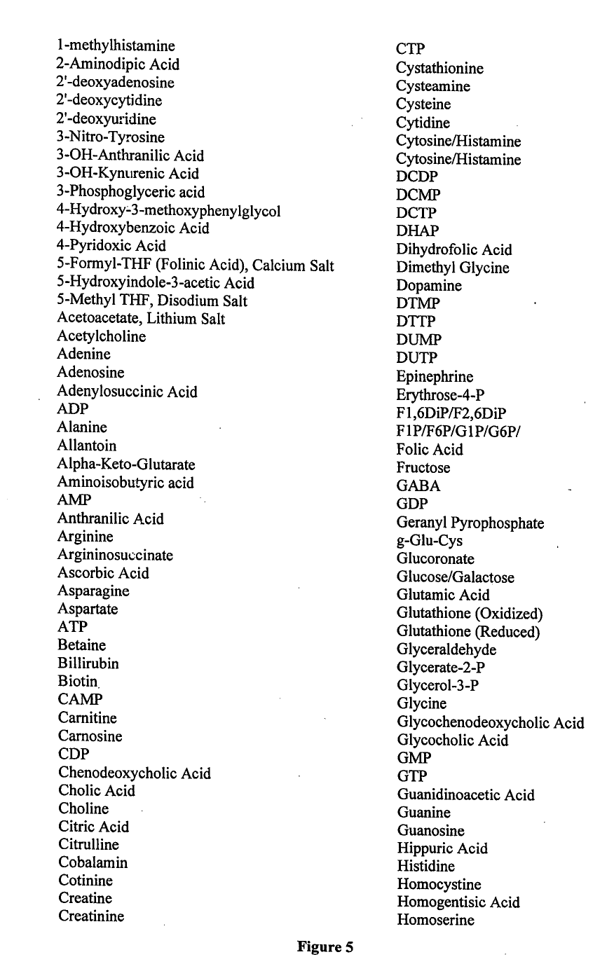

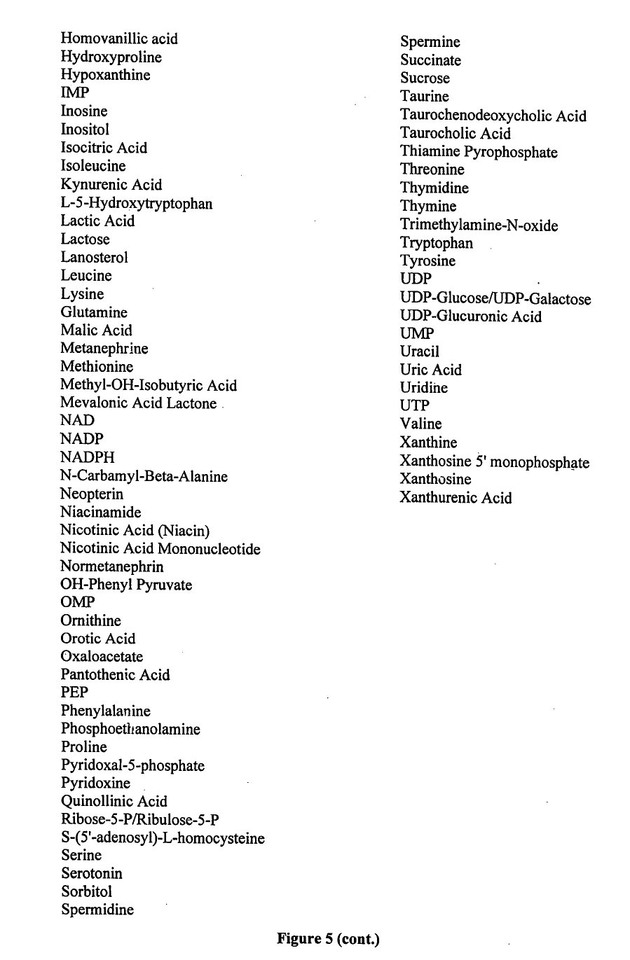

173 of the analytes assayed were known, having been evaluated by high accuracy mass spectrometry in studies using purified compounds spiked into plasma across a range of concentrations. In prior studies, the coefficient of variation at the typical circulating plasma concentrations was <10% in 25% of the analytes, 10-20% in 35% of the analytes, 20-30% in 20% of the analytes and >30% in the remainder. Some of the low molecular weight peaks seen reproducibly in human plasma have not yet been unambiguously identified, and are designated as such by the prefix MET. A list of the known metabolites analyzed is included in FIG. 5.

E. Statistical Analysis

Metabolites for which the distribution of the log-transformed levels in the study population had absolute values of skew and kurtosis <1 and a non-significant Wilkes-Shapiro test were deemed to have a normal distribution and analyzed using parametric tests; metabolites for which the distribution failed to meet these criteria were analyzed using non-parametric tests. The significance of the change in log-transformed metabolite levels from pre-test to post-test was assessed using paired Student's t-tests or Wilcoxon signed-rank tests, as appropriate. Changes in metabolites are expressed as percent increases or decreases from the untransformed baseline levels and, for the sake of consistency, medians and interquartile ranges (IQR) are used for all metabolites. To compare the correlation between log-transformed metabolite levels and degree of exertion or extent of ischemic myocardium, correlation coefficients were calculated.

F. Metabolic Profiling Results

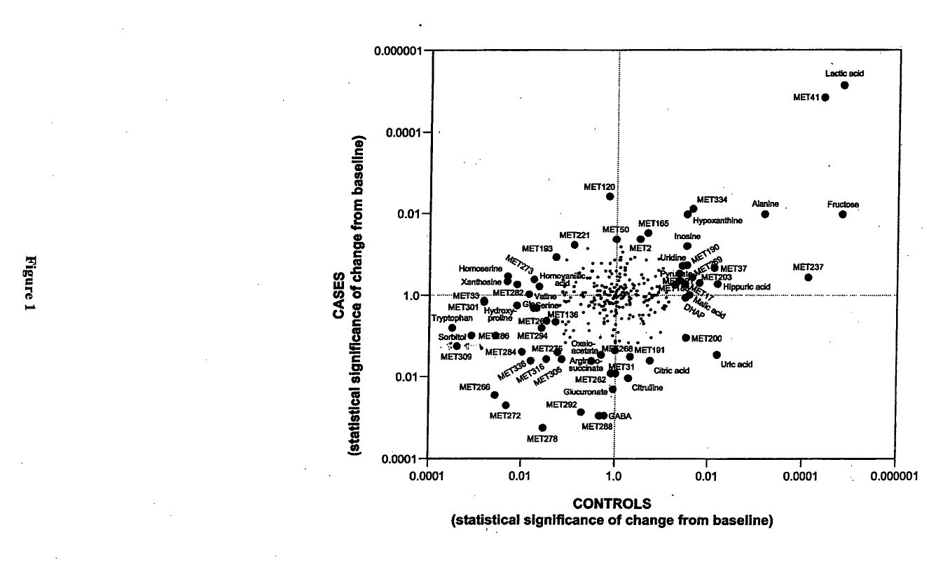

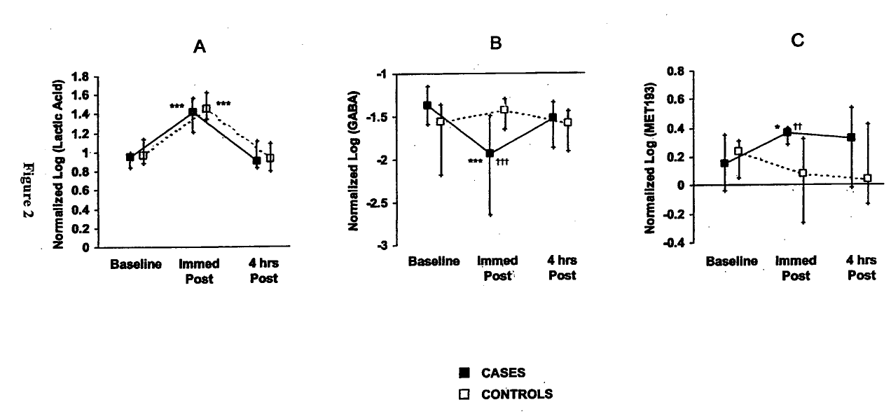

For each metabolite, the statistical significance of the change in the circulating level from immediately before exercise to immediately after exercise was calculated separately in cases and controls. The results are plotted in FIG. 1, in which the position on the X-axis represents the statistical significance of the change in controls, and the position on the Y-axis represents the statistical significance of the change in cases. Metabolites on the right half of the scatterplot increased in controls after stress testing, whereas metabolites on the left half decreased. Similarly, metabolites on the top half of the scatterplot increased in cases, whereas metabolites on the bottom half decreased.

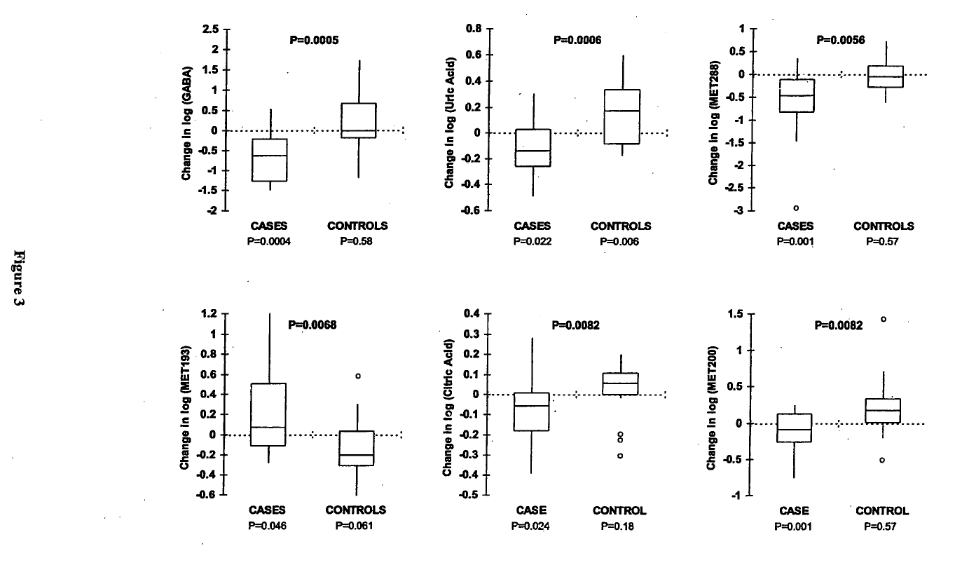

The majority of metabolites displayed concordant changes in cases and controls (i.e., increased in both or decreased in both). The upper right quadrant of FIG. 1 contains metabolites that increased in both cases and controls. For example, immediately after exercise, median levels of lactic acid, an end product of glycolysis when the amount of oxygen is limiting, increased by 177% (Interquartile range (IQR) 105 to 257%; P<0.0001). The changes observed after exercise were similar in cases and controls (FIG. 2A) and resolved by four hours after exercise. Similarly, median levels of metabolites involved in skeletal muscle adenosine monophosphate catabolism increased after exercise in both cases and controls (upper right quadrant of FIG. 1). These included hypoxanthine (46%, IQR −8 to 106%, P=0.0004) and inosine (67%, IQR −18 to 175%, P=0.003). In addition; median levels of alanine, a nitrogen transporter exported by skeletal muscle, also increased after exercise in cases and controls (19%, IQR 2 to 35%, P<0.0001).

Metabolites demonstrating discordant regulation between cases and controls were subsequently examined. As seen at the bottom center of FIG. 1, plasma levels of GABA and MET 288 decreased strikingly in cases (−77%, IQR −37 to −94%, P=0.0004 and −65%, IQR −23 to −85%, P=0.001, respectively), but remained unchanged in controls. The levels of GABA in cases and controls are shown over time in FIG. 2B, which illustrates how levels returned to baseline in cases by four hours. Significant decreases were also observed in the levels of oxaloacetate (−25%, IQR 5 to −39%, P=0.023), citrulline (−25%, IQR 2 to −36%, P=0.009), and argininosuccinate (73%, IQR 25 to −84%, P=0.012) in cases only. Both oxaloacetate (r=−0.65, P=0.0035) and citrulline (r=−0.46, P=0.054) exhibited moderately strong trends toward correlating with the extent of the perfusion defect during stress testing.

As seen in the lower right quadrant of FIG. 1, three metabolites were significantly differentially regulated in cases (decreased) and controls (increased) including uric acid (P=0.0006), citric acid (P=0.008), and MET200 (P=0.008). Conversely, MET193 (P=0.0068) and MET 221 (P=0.01) increased in cases (with the changes in MET193 persisting through four hours after the ischemic insult, FIG. 2C), but decreased in controls (upper left of FIG. 1). Of note, in this small clinical cohort there was no evidence of significant heterogeneity in the magnitude of the changes in metabolites in cases with and without diabetes, hyperlipidemia, heart failure, or peripheral arterial disease, consistent with the notion that the changes are due to myocardial ischemia rather than cardiac risk factors.

G. Functional Pathway Analysis

To determine whether the observations described herein of changes in individual metabolites in the setting of myocardial ischemia, in fact, reflected coordinate changes in defined metabolic pathways, software was developed to identify functional or pathway trends. This software was based on FuncAssociate, originally designed to reveal pathway trends in high-throughput mRNA expression data. (Berriz, G. F., et al. 2003 Bioinformatics 19:2502-4; as well as Harvard University's Roth Computational Biology Laboratory's FuncAssociate program, which takes lists of genes as input and produces a ranked list of Gene Ontology attributes that the input list is enriched or depleted for.)

Metabolites were characterized using attributes from the KEGG database (the Kyoto Encyclopedia of Genes and Genomes bioinformatics resource, part of the research projects run in the Kanehisa Laboratory of Kyoto University Bioinformatics Center). These attributes are of the form “participates in reaction R”, or “participates in pathway P”, or “is associated with human disease D”. Attributes were used that were associated with at least 3 metabolites. The total number of attributes examined was 96. The metabolites were ranked as follows: for every metabolite, a Wilcoxon rank-sum test was applied; for controls, a one sample test was used against the null hypothesis of zero exercise-related change in the metabolite; for cases, a two sample test was used against the null hypothesis that ischemic patients and control patients had the same exercise-related response.

The metabolites were then sorted by the signed-significance of each test, respectively. Signed-significance is defined as the negative of the log (base 10) of the test p-value, multiplied by the sign of the median (in the case of the exercise list) or the difference in medians between the two samples (in the case of disease list).

For the subsequent analysis, the unknown metabolites were discarded. Thus, two ranked lists of metabolites were generated, one for cases and one for controls. For each of these two ranked lists of metabolites, together with the same lists in reverse order (four ranked lists in total), a cumulative hypergeometric test (Fisher's Exact Test) was used at each possible rank threshold to score attributes of these metabolites according to their degree of overrepresentation among metabolites above the rank threshold. Specifically, for each metabolite attribute A and each “initial k-sublist” of metabolites (which is the ranked list of metabolites consisting of the first k metabolites in the original ranked list), the Fisher's exact test P value was computed for the categorical variables “belongs to initial k-sublist” and “has attribute A”. The k-sublist with the smallest P-value was assigned to each attribute, and the attributes were ranked in ascending order by this P-value.

For each ranked list of metabolites, this analysis was repeated 1,000 times using random permutations of the original ranked metabolite list as input. The null hypothesis for each ranked list was that no metabolite attribute is more enriched among the top-ranked metabolites than would be expected from a randomly-ranked list of metabolites. To limit type I error, the multiple-hypothesis-corrected (adjusted) P-value for a given metabolite attribute is the fraction of random control runs with an unadjusted P-value (for any metabolite attribute) less than or equal to the observed unadjusted P-value for the metabolite attribute of interest. (For example, if the unadjusted P-value was 0.002 and the adjusted P-value was 0.01, this means that after generating 1000 random permutations of the data, the fraction of permutations in which the unadjusted P-value was <0.002 was 0.01.). This procedure has been described elsewhere in detail (Berriz, G. F., et al. 2003 Bioinformatics 19:2502-4).

Analysis of all the known metabolites in the dataset generated revealed that members of the citric acid pathway were significantly over-represented in the list of metabolites that changed specifically in the setting of myocardial ischemia, with 6 members of the citric acid cycle pathway falling within the top 23 most-changed metabolites (P=0.00031, P=0.04 after adjusting for multiple testing).

H. Ischemia Risk Score

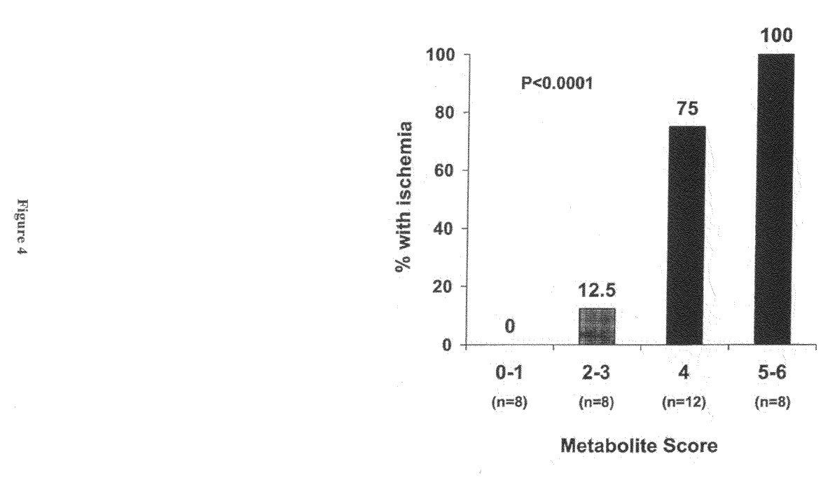

Based on these observations, it was subsequently investigated whether metabolic profiling could be used to accurately distinguish patients with ischemia from those without. Differences between the change (pre- to post-test) in a metabolite in cases vs. controls having been compared using Wilcoxon rank-sum tests, cutpoints were selected using receiver-operator characteristic (ROC) curve analysis to maximize accuracy for metabolites that displayed significantly discordant regulation in cases vs. controls (<0.01) (6 metabolites, FIG. 3). A metabolic (ischemia) risk score was computed by assigning patients one point for each metabolite for which the change exceeded the cutpoint for ischemia (FIG. 4).

To estimate the degree of optimism in the discriminatory ability of our score, six-fold cross-validation was performed (Stone, M. 1974 J of the Royal Stat Soc. Ser B 36:111-147; Efron, B, et al. 1983 The American Statistician 37:3648). The dataset was randomly divided into six subsets, each subset containing three cases and three controls. Using the methodology described above, a metabolic score was developed in a training set containing 5 subsets. This score was then validated in a testing set consisting of the remaining withheld subset. This process was repeated so that each subject in the dataset was used in one testing set. The c-statistics in each testing set were then averaged to provide a cross-validated c-statistic. The score yielded a highly statistically significant relationship to the probability of ischemia (P<0.0001) as well as excellent discrimination (c-statistic 0.95). The preserved discriminatory ability had a c-statistic of 0.83.

In summary, it was postulated that perturbations that arise either as a cause or consequence of disease may be detected as particular patterns of metabolites or proteins in the blood. To that end, metabolomics have been applied to myocardial ischemia in a carefully characterized cohort of 36 patients undergoing exercise stress testing. Using state-of-the-art metabolic profiling, significant changes have been demonstrated herein after exercise stress testing in circulating levels of multiple metabolites. Distinct clusters of related metabolites have been identified that demonstrated coordinate responses to either exercise in some cases or to ischemia in others. Finally, metabolic profiling was employed to differentiate patients who developed inducible ischemia from those who did not with a high degree of accuracy.

An important rationale for unequivocally identifying analytes or surveying known analytes, is to gain insight into the functionally relevant cellular mechanisms contributing to disease pathways. Having hundreds of named metabolites allowed the identification herein of multiple participants in particular biological pathways moving in tandem, which enhanced confidence that individual participants in that pathway were truly correlated with the perturbation. In principle, incorporating knowledge of pathways into candidate marker triage, increases the likelihood that selected biomarkers will be validated in subsequent prospective studies. The use of pathway analysis should also prove advantageous in ongoing efforts to identify novel peaks using recently developed techniques such as Fourier transform mass spectrometry.

### Example 2

### Planned Myocardial Infarction (MI)

### Changes in Metabolite Levels Before Vs. after Alcohol Septal Ablation

Although the steep time-to-treatment benefit curve of current reperfusion strategies for Acute Myocardial Infarction (MI) mandates prompt diagnosis, serum markers of MI have limited sensitivity and specificity in the initial minutes to hours following the onset of injury. Recent advances in metabolic profiling technologies have enhanced the feasibility of high throughput patient screening for the diagnosis of disease states. Here, liquid chromatography was applied with high sensitivity electrospray mass spectrometry to assay 470 metabolites in patients undergoing alcohol septal ablation for hypertrophic cardiomyopathy, a human model of “Planned” MI (PMI). This model is particularly powerful as serial sampling can be performed in patients before and after a controlled myocardial insult, thereby allowing each patient to serve as his or her own biological control.

The following example shows that the septal ablation model does, indeed, recapitulate important features of clinical MI, including typical echocardiographic and electrocardiographic ST changes (data not shown).

A. Patient Enrollment

Protocol approval was obtained from the Massachusetts General Hospital IRB for patient enrollment and sample collection. Inclusion criteria for patients undergoing septal ablation included: 1) patients with primary hypertrophic cardiomyopathy 2) septal thickness of 16 mm or greater 3) resting outflow tract gradient of greater than 30 mmHg, or an inducible outflow tract gradient of greater than 50 mm Hg 4) symptoms refractory to optimal medical therapy and 5) appropriate coronary anatomy. Additional data were obtained on each patient's age, sex, race, weight, and cardiac risk factors.

B. Ablation Protocol

For septal ablations, left heart catheterization was performed by the transseptal technique. The most proximal accessible septal branch was instrumented using standard angioplasty guiding catheters and guidewires and 1.5 or 2.0 mm×9 mm Maverick™ balloon catheters. Radiographic and echocardiographic contrast injections confirmed proper selection of the septal branch and balloon catheter position (Brindle, J. T. et al. 2002 Nat Med 8:143944; Fiehn, O., et al. 2000 Nat Biotechnol 18:1157-61). Ethanol was infused through the balloon catheter at 1 ml per minute. Additional injections in the same or other septal branches were administered as needed to reduce the gradient to <20 mmHg. Mean ethanol dosage was 2.8±1.9 ml.

For the ablation “Planned” MI (PMI) study, 35 patients in total were enrolled. 28 patients were randomly selected for the derivation set and 7 for the validation set. In 9 patients, simultaneous coronary sinus and femoral samples were obtained. For the “normal” MI validation cohort, patients were enrolled who presented to the cardiac catheterization suite with either acute ST elevation MI (n=12 patients) or a control group with stable coronary artery disease (n=10 patients).

C. Sample Procurement

For septal ablation patients, blood was drawn at baseline, just prior to the alcohol injection, and at 10 minute, one hour, two hour and 24 hour time points, from femoral and/or coronary sinus catheters, into chilled citrated tubes (Becton Dickinson, 0.105M buffered sodium citrate, whole blood ratio 1:9). Coronary sinus samples were obtained at the baseline, 10 minute and 1 hour timepoints; the coronary sinus catheter was subsequently removed prior to the patient leaving the catheterization suite.

For the validation cohort, one sample was obtained from a femoral venous catheter during the procedure. Samples were centrifuged at 2000×g for 10 min to pellet cellular elements. The supernatant plasma was then aliquoted, immediately frozen and preserved at −80° C. to minimize freeze-thaw degradation. In parallel with each experimental draw, an additional blood sample was sent to the clinical laboratory for evaluation of the standard cardiac markers CK (with MB fraction) and TnT (Roche Diagnostics).

Creatine Kinase (CK) and Troponin T, the standard biochemical metrics of myocardial injury, appeared in the plasma of septal ablation patients in a time course consistent with “normal” MI (data not shown).

To further validate the human model and the robustness of the metabolomics platform, the responses of specific metabolites were examined which were expected to be modulated following acute myocardial injury.

D. HPLC and Mass Spectrometry Analysis

All samples were handled in a blinded fashion, with the order of processing randomized. Amino acids and amines were separated by reverse phase chromatography on a Luna phenyl-hexyl column (Phenomenex, Torrance, Calif.) using acetonitrile/water/0.1% acetic acid at pH 3.5-4.0 in a run time of 1.5 minutes. Sugars and ribonucleotides were separated on a Luna amino column (Phenomenex, Torrance, Calif.) under normal phase using acetonitrile/water/0.25% ammonium hydroxide/10 mM ammonium acetate at pH 11 in a run time of 3.5 minutes.

Organic acids were separated using a Synergi Polar-RP column (Phenomenex, Torrance, Calif.) under reverse phase using acetonitrile/water/5 mM ammonium acetate at pH 5.6-6.0 in a run time of 3.5 minutes. Columns were connected in parallel via an automated switching valve on a robotic sample loader (Leap Technologies). A triple quadrupole mass spectrometer (API4000, Applied Biosystem/Sciex) was operated in an automated switching polarity mode using a turbo ion spray LC/MS interface under selected reaction monitoring (SRM) conditions. A total of 470 parent/daughter (P/D) ion pairs were monitored through six SRM experiments on each sample.

Known metabolites had previously been evaluated using high accuracy mass spectrometry of the purified compound spiked into plasma across a range of concentrations. In these prior studies, the coefficient of variation at typical circulating plasma concentrations was <10% in 25% of the analytes, 10-20% in 35% of the analytes, 20-30% in 20% of the analytes and >30% in the remainder. Unknown metabolites, designated by the prefix MET or CAP, are low molecular weight peaks that are reproducibly seen in human plasma but have not yet been unambiguously identified. Metabolite quantification was performed by integrating peak areas for each parent/daughter pairs, and normalized to an internal standard to account for variations in injection volume.

E. Statistical Analysis

Levels of metabolites were tested for statistically significant change from baseline using either the Wilcoxon signed rank test, or the paired T-test for those metabolites which were normally distributed. Metabolites were considered to be normally distributed only if distributions at each time point were all normal, based on a non-significant Wilkes-Shapiro Test. Comparisons between any two time points included only those patients with samples available for both time points. A p-value <0.05 was considered statistically significant. Data in all figures represent absolute values, and are presented with medians and interquartile ranges (IQR).

### Example 3

### Composite Biomarkers

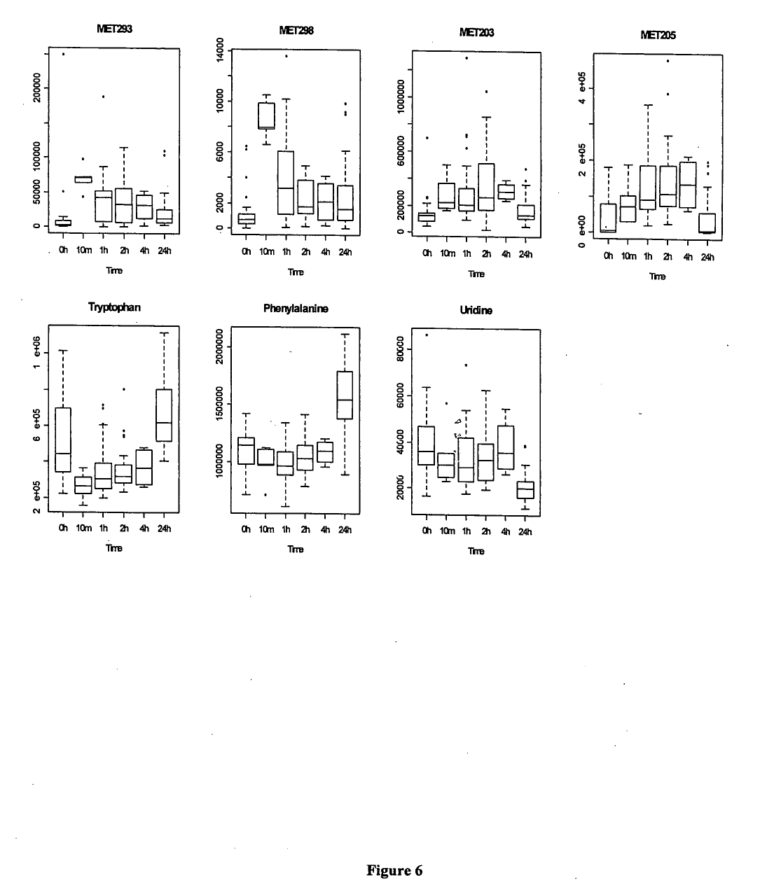

Peripheral blood samples were then assessed across the range of time points available to generate candidates for early (10 minute), intermediate (1-4 hour) and late (24 hour) metabolomic biomarkers of “planned” myocardial injury. For each time point, metabolites were ranked by the statistical significance of their change (either increased or decreased), as compared to baseline values. The two most changed metabolites at each time point that also demonstrated persistence of change for at least one prior or subsequent time point were then selected as candidate biomarkers. Because several metabolites were among the most significantly changed at multiple time points, this process yielded seven markers in total: three known metabolites (Tryptophan, Phenylalanine, and Uridine), as well as four metabolites (ME7293, MET298, MET203, MET205) which had previously not been unambiguously identified, (FIG. 6). In the meantime, ME1205 (or, CAP205) has been identified as hydroxyhippuric acid, and MET203 (or, CAP203) has been identified as aconitic acid.

A “composite biomarker” (Comp7) was formed to capture the complementary strengths of the individual biomarkers. Comp7 represents a simple linear combination of each marker weighted such that an equivalent percentage change in the level of different metabolites would contribute to a similar degree to a change in Comp7.

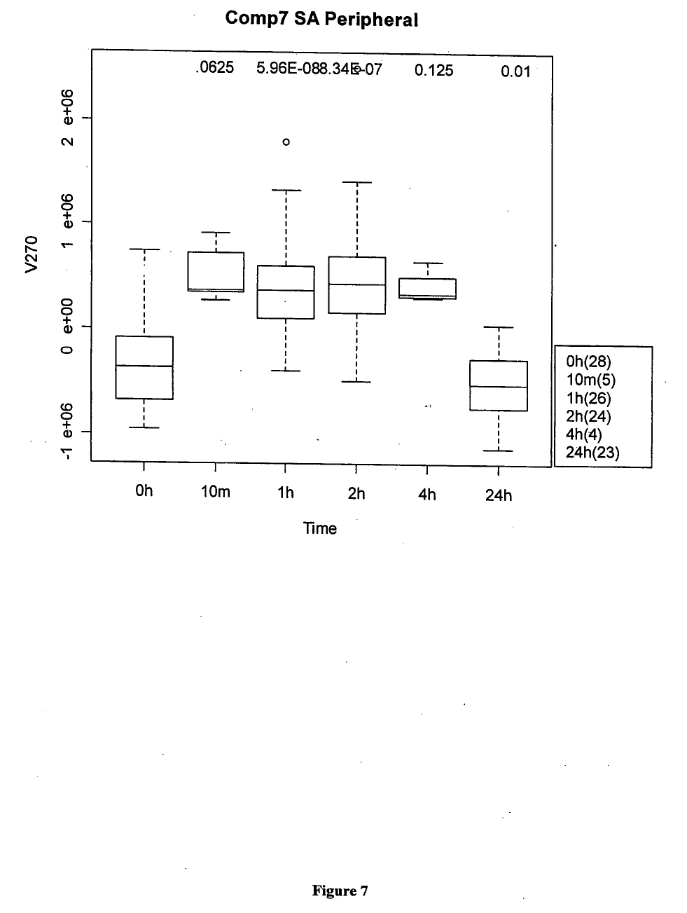

For the initial septal ablation training set of 26 patients, as early as 10 minutes after alcohol septal ablation, the Comp7 level in peripheral blood rose acutely and stayed elevated past 4 hours, before returning to baseline by 24 hours (FIG. 7). An arbitrary Comp7 threshold value of “0” appeared to separate pre- from post-infarct values. Although the limited number of samples preclude statistical validation at the 10 minute and 4 hour timepoints, the trend suggests impressive overall potential as an early marker of myocardial injury that persists over several hours.

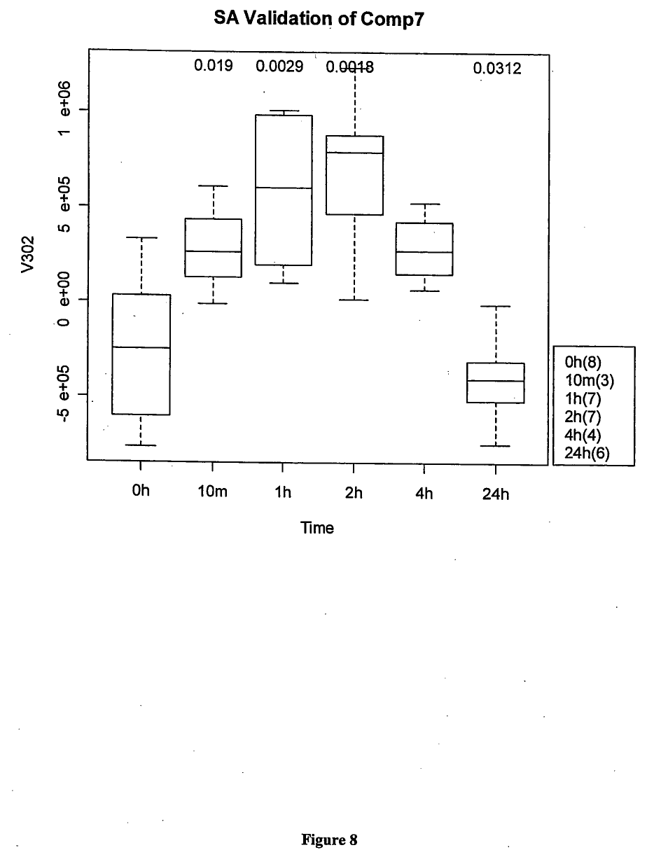

Because all biomarker discovery studies are vulnerable to unintentional “overfitting” of data, the performance of Comp7 was next assessed in a second, independent group of patients undergoing alcohol septal ablation. The discriminatory ability of Comp7 for myocardial injury was found to be equally powerful and reproducible, even with the smaller sample size of the validation set. Importantly, Comp7 detected the presence of myocardial injury in samples where no significant rise in cardiac troponin was noted (at the 10 minute timepoint; FIG. 8).

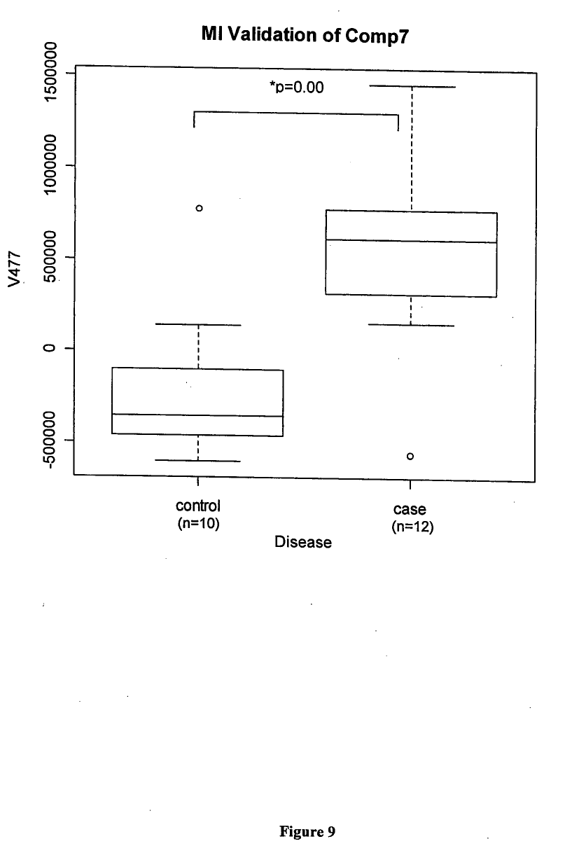

To test whether these results might translate into the clinical arena, Comp7 was applied to samples from patients presenting to the cardiac catheterization suite with either stable coronary artery disease or “normal” acute ST elevation MI, as assessed by electrocardiographic changes and ultimately troponin release. Samples from the acute MI group were collected within the first 4 hours of onset of symptoms, which falls within the time interval of expected Comp7 elevation. The measured mean peak CPK in these infarcts was 3059±1894, with a peak Troponin-T of 10.33±5.93. Upon plotting Comp7 levels from patients with myocardial infarcts (“cases”) as compared to “controls”, the majority of controls were found to have Comp7 values below zero; conversely, the majority of cases exhibited highly positive Comp7 levels (P=0.003), as seen in FIG. 9. Thus, the composite biomarker derived in “Planned MI” was validated in real world samples, as well.

In summary, in an effort to test the concept that perturbations that arise either as a cause or consequence of disease may be detected as particular patterns of metabolites or proteins in the blood, the novel application of metabolomics to a carefully characterized cohort of patients undergoing planned myocardial injury has been presented herein. Significant changes were demonstrated in circulating levels of multiple metabolites after planned injury, including those in adenine and tryptophan metabolism defecting hypoxic and inflammatory changes following myocardial injury. Biomarkers derived in the PMI cohort were subsequently validated in distinct clinical cohorts, diagnosing myocardial injury as early as within 10 minutes of onset. These findings were also reproduced in a pig model of regional ischemia, and attest to common biochemical pathways of energy metabolism across species. Furthermore, simultaneously drawn coronary sinus samples suggest cardiac-specific enrichment of several of the metabolites.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

1. A method of detecting myocardial ischemia or myocardial infarction in a subject comprising detecting a change in the amount of at least one member of the group consisting of lactic acid, hypoxanthine, inosine, alanine, GABA, oxaloacetate, citrulline, argininosuccinate, uric acid, citric acid, uridine, phenylalanine, tryptophan, serine, hydroxyhippuric acid, aconitic acid, and a metabolic product thereof in a biological sample obtained from the subject, thereby detecting myocardial ischemia or early myocardial infarction in the subject.

2. The method of claim 1, wherein the change comprises a decrease in the amount of at least one member of the group consisting of GABA, oxaloacetate, citrulline, argininosuccinate, uric acid, citric acid, tryptophan, serine, uridine, and a metabolic product thereof.

3. The method of claim 1, wherein the change comprises an increase in the amount of at least one member of the group consisting of lactic acid, hypoxanthine, inosine, alanine, phenylalanine, hydroxyhippuric acid, aconitic acid, and a metabolic product thereof.

4. The method of claim 3, wherein the change comprises an increase in the amount of at least one member of the group consisting of hydroxyhippuric acid, aconitic acid, and a metabolic product thereof.

5. The method of claim 1, wherein the change comprises an increase in the amount of at least one member of the group consisting of lactic acid, hypoxanthine, inosine, alanine, phenylalanine, hydroxyhippuric acid, aconitic acid, and a metabolic product thereof and a decrease in at least one member of the group consisting of GABA, oxaloacetate, citrulline, argininosuccinate, uric acid, citric acid, tryptophan, serine, uridine, and a metabolic product thereof.

6. The method of claim 1, wherein myocardial infarction is detected.

7. The method of claim 6, wherein the myocardial infarction is early myocardial infarction.

8. The method of claim 6, wherein the change comprises an increase in the amount of at least one member of the group consisting of lactic acid, hypoxanthine, inosine, alanine, phenylalanine, hydroxyhippuric acid, aconitic acid, and a metabolic product thereof and a decrease in at least one member of the group consisting of GABA, citric acid, oxaloacetate, citrulline, argininosuccinate, uric acid, tryptophan, serine, uridine, and a metabolic product thereof.

9. The method of claim 8, wherein the change comprises an increase in the amount of at least one member of the group consisting of hydroxyhippuric acid, aconitic acid, and a metabolic product thereof and a decrease in at least one member of the group consisting of GABA, citric acid, oxaloacetate, citrulline, argininosuccinate, uric acid, tryptophan, serine, uridine, and a metabolic product thereof.

10. The method of claim 1, wherein myocardial ischemia is detected.

11. The method of claim 10, wherein the change comprises an increase in the amount of at least one member of the group consisting of lactic acid, hypoxanthine, inosine, alanine, hydroxyhippuric acid, and a metabolic product thereof and a decrease in the amount of at least one member of the group consisting of GABA, oxaloacetate, citrulline, argininosuccinate, uric acid, citric acid, and a metabolic product thereof.

12. The method of claim 11, wherein the change comprises an increase in the amount of at least one member of the group consisting of hydroxyhippuric acid and a metabolic product thereof and a decrease in at least one member of the group consisting of GABA, citric acid, oxaloacetate, citrulline, argininosuccinate, uric acid, and a metabolic product thereof.

13. The method of claim 1, wherein the biological sample comprises a blood sample or a preparation thereof.

14. The method of claim 13, wherein the preparation comprises plasma or serum.

15. The method of claim 1, wherein the subject is a human.

16. The method of claim 1, wherein the change is detected after administration of a controlled ischemic insult or planned myocardial infarction to the subject.

17. The method of claim 16, wherein the controlled ischemic insult comprises exercise testing, and the planned myocardial infarction comprises alcohol septal ablation for hypertrophic cardiomyopathy.

18. The method of claim 1, wherein the detecting comprises analyzing the sample, or a preparation thereof, using liquid chromatography and mass spectrometry.

19. The method of claim 18, wherein the mass spectrometry comprises high sensitivity electrospray mass spectrometry.

20. A metabolic profile indicating myocardial ischemia or myocardial infarction in a subject comprising a change in the amount of at least one member of the group consisting of lactic acid, hypoxanthine, inosine, alanine, GABA, oxaloacetate, citrulline, argininosuccinate, uric acid, citric acid, uridine, phenylalanine, tryptophan, serine, hydroxyhippuric acid, aconitic acid, and a metabolic product thereof in a biological sample obtained from the subject.

21. The profile of claim 20, wherein the change comprises a decrease in the amount of at least one member of the group consisting of GABA, oxaloacetate, citrulline, argininosuccinate, uric acid, citric acid, tryptophan, serine, uridine, and a metabolic product thereof.

22. The profile of claim 20, wherein the change comprises an increase in the amount of at least one member of the group consisting of lactic acid, hypoxanthine, inosine, alanine, phenylalanine, hydroxyhippuric acid, aconitic acid, and a metabolic product thereof.

23. The profile of claim 22, wherein the change comprises an increase in the amount of at least one member of the group consisting of hydroxyhippuric acid, aconitic acid, and a metabolic product thereof.

24. The profile of claim 20, wherein the change comprises an increase in the amount of at least one member of the group consisting of lactic acid, hypoxanthine, inosine, alanine, phenylalanine, hydroxyhippuric acid, aconitic acid, and a metabolic product thereof and a decrease in the amount of at least one member of the group consisting of GABA, oxaloacetate, citrulline, argininosuccinate, uric acid, citric acid, tryptophan, serine, uridine, and a metabolic product thereof.

25. The profile of claim 20, wherein myocardial infarction is indicated.

26. The profile of claim 25, wherein the myocardial infarction is early myocardial infarction.

27. The profile of claim 25, wherein the change comprises an increase in the amount of at least one member of the group consisting of lactic acid, hypoxanthine, inosine, alanine, phenylalanine, hydroxyhippuric acid, aconitic acid, and a metabolic product thereof and/or a decrease in the amount of at least one member of the group consisting of GABA, citric acid, oxaloacetate, citrulline, argininosuccinate, uric acid, tryptophan, serine, uridine, and a metabolic product thereof.

28. The profile of claim 27, wherein the change comprises an increase in the amount of at least one member of the group consisting of hydroxyhippuric acid, aconitic acid, and a metabolic product thereof and a decrease in at least one member of the group consisting of GABA, citric acid, oxaloacetate, citrulline, argininosuccinate, uric acid, tryptophan, serine, uridine, and a metabolic product thereof.

29. The profile of claim 20, wherein myocardial ischemia is indicated.

30. The profile of claim 29, wherein the change comprises an increase in the amount of at least one member of the group consisting of lactic acid, hypoxanthine, inosine, alanine, hydroxyhippuric acid, and a metabolic product thereof and/or a decrease in the amount of at least one member of the group consisting of GABA, oxaloacetate, citrulline, argininosuccinate, uric acid, citric acid, and a metabolic product thereof.

31. The method of claim 30, wherein the change comprises an increase in the amount of at least one member of the group consisting of hydroxyhippuric acid and a metabolic product thereof and a decrease in at least one member of the group consisting of GABA, citric acid, oxaloacetate, citrulline, argininosuccinate, uric acid, and a metabolic product thereof.

32. The profile of claim 20, wherein the biological sample comprises a blood sample or a preparation thereof.

33. The profile of claim 32, wherein the preparation comprises plasma or serum.

34. The profile of claim 20, wherein the subject is a human.

35. The profile of claim 20, wherein the change results from administration of a controlled ischemic insult or planned myocardial infarction to the subject.

36. The profile of claim 35, wherein the controlled ischemic insult comprises exercise testing, and the planned myocardial infarction comprises alcohol septal ablation for hypertrophic cardiomyopathy.

37. A method of obtaining a metabolic profile of a subject afflicted with, or at risk of becoming afflicted with, myocardial ischemia, comprising the steps of:
i) analyzing a biological sample obtained from the subject; and
ii) detecting a change in the amount of at least one member of the group consisting of lactic acid, hypoxanthine, inosine, alanine, GABA, oxaloacetate, citrulline, argininosuccinate, uric acid, citric acid, hydroxyhippuric acid, and a metabolic product thereof, thereby obtaining a metabolic profile of a subject afflicted with, or at risk of becoming afflicted with, myocardial ischemia.

38. The method of claim 37, wherein the change is in the amount of at least one member of the group consisting of hydroxyhippuric acid and a metabolic product thereof.

39. The method of claim 37, wherein the biological sample is obtained from the subject before and after subjecting the subject to controlled ischemic insult.

40. The method of claim 39, wherein the controlled ischemic insult comprises exercise testing.

41. A method of obtaining a metabolic profile of a subject afflicted with, or at risk of becoming afflicted with, myocardial infarction, comprising the steps of:
i) analyzing a biological sample obtained from the subject; and
ii) detecting a change in the amount of lactic acid, hypoxanthine, inosine, alanine, GABA, oxaloacetate, citrulline, argininosuccinate, uric acid, citric acid, uridine, phenylalanine, tryptophan, serine, hydroxyhippuric acid, aconitic acid, and a metabolic product thereof, thereby obtaining a metabolic profile of a subject afflicted with, or at risk of becoming afflicted with, myocardial infarction.

42. The method of claim 41, wherein the change is in the amount of at least one member of the group consisting of hydroxyhippuric acid, aconitic acid, and a metabolic product thereof.

43. The method of claim 41, wherein the myocardial infarction is early myocardial infarction.

44. The method of claim 41, wherein the biological sample is obtained before and after subjecting the subject to planned myocardial infarction.

45. The method of claim 44, wherein the planned myocardial infarction comprises alcohol septal ablation for hypertrophic cardiomyopathy.

46. The method of claim 37, wherein the biological sample comprises a blood sample or preparation thereof.

47. The method of claim 46, wherein the preparation comprises plasma or serum.

48. The method of claim 37, wherein the subject is a human.

49. The method of claim 37, wherein the analyzing comprises subjecting the sample, or a preparation thereof, to liquid chromatography and mass spectrometry.

50. The method of claim 49, wherein the mass spectrometry comprises high sensitivity electrospray mass spectrometry.

51. A method of identifying a metabolic biomarker for myocardial ischemia, comprising the steps of:
i) obtaining a biological sample from a subject before and after subjecting the subject to controlled ischemic insult;
ii) analyzing the samples for changes in amounts of metabolic products; and
iii) identifying the metabolic products,
thereby identifying a metabolic biomarker for myocardial ischemia.

52. The method of claim 51, wherein the controlled ischemic insult comprises exercise testing.

53. A method of identifying a metabolic biomarker for myocardial infarction, comprising the steps of:
i) obtaining a biological sample from a subject before and after subjecting the subject to planned myocardial infarction;
ii) analyzing the samples for changes in amounts of metabolic products; and
iii) identifying the metabolic products,
thereby identifying a metabolic biomarker for myocardial infarction.

54. The method of claim 53, wherein the myocardial infarction is early myocardial infarction

55. The method of claim 53, wherein the planned myocardial infarction comprises alcohol septal ablation for hypertrophic cardiomyopathy.

56. The method of claim 51, wherein the biological sample comprises a blood sample or preparation thereof.

57. The method of claim 56, wherein the preparation comprises plasma or serum.

58. The method of claim 51, wherein the subject is a human.

59. The method of claim 51 wherein the analyzing comprises subjecting the sample, or a preparation thereof, to liquid chromatography and mass spectrometry, and wherein the identifying comprises comparing the mass spectra obtained with those of known metabolic products.

60. The method of claim 59, wherein the mass spectrometry comprises high sensitivity electrospray mass spectrometry.

61. The method of claim 41, wherein the biological sample comprises a blood sample or preparation thereof.

62. The method of claim 41, wherein the subject is a human.

63. The method of claim 41, wherein the analyzing comprises subjecting the sample, or a preparation thereof, to liquid chromatography and mass spectrometry.

64. The method of claim 53, wherein the biological sample comprises a blood sample or preparation thereof.

65. The method of claim 53, wherein the subject is a human.

66. The method of claim 53, wherein the analyzing comprises subjecting the sample, or a preparation thereof, to liquid chromatography and mass spectrometry, and wherein the identifying comprises comparing the mass spectra obtained with those of known metabolic products.
